(19) United States
(12) Patent Application Publication
(10) Pub. No.: US 2015/0320928 A1
(43) Pub. Date: Nov. 12, 2015

(54) SURGICAL PLATFORM FOR EXTREMITIES

(71) Applicant: Timothy Graham Allen, Frisco, TX (US)

(72) Inventor: Timothy Graham Allen, Frisco, TX (US)

(21) Appl. No.: 14/804,580

(22) Filed: Jul. 21, 2015

Related U.S. Application Data
(63) Continuation-in-part of application No. 13/947,494, filed on Sep. 22, 2013.

(51) Int. Cl. A61M 3/02; A61G 13/12; A61B 19/00

(52) U.S. Cl. CPC A61M 3/0287; A61B 19/38; A61B 19/42; A61G 13/124; A61M 2210/083

(57) ABSTRACT

Platforms for irrigating, debriding, treating, and operating on wounds and infections in extremities are provided.

### CROSS-REFERENCE TO RELATED APPLICATIONS

This application is a continuation-in-part of, and claims benefit of priority to, U.S. patent application Ser. No. 13/947,494, filed in the United States Patent and Trademark Office on Sep. 22, 2013, the content of which is incorporated herein by reference in its entirety.

### BACKGROUND OF THE DISCLOSURE

Debriding and washing wounds and infections in extremities such as arms and hands are common procedures in operating rooms. During irrigation or lavage processes, an extremity should be supported properly to prevent further injury, to reduce contamination issues, and to capture irrigation and/or bodily fluid and debris rather than draining haphazardly onto an operating table or floor. Some devices attempt to support a limb during irrigation while capturing fluid drainage, but these are cumbersome, complex and have limited utility. Some devices, for instance, require assembly of a pan and a cover with support structures between the pan and cover to support the weight of a limb on the cover. Typically, the cover is a flat area for the lavage process while the pan is for fluid collection and not for surgical procedures thereon.

What is lacking and therefore needed in the medical industry is a lightweight, portable, multipurpose device that can be used for debridement and lavage processes as well as for surgical procedures.

### BRIEF SUMMARY OF THE DISCLOSURE

The present disclosure is directed in general to irrigation basins or platforms with opposing sides or halves and other features having a variety of purposes. The various embodiments of the platforms described herein generally accommodate a human hand and wrist, for instance, so that a traumatic or surgical wound can be safely and neatly irrigated to cleanse an extremity or appendage of blood, tissue, soilage, infection, and/or other contamination or debris. An exemplary platform safely secures the hand and wrist during a procedure and protects the sterile field during irrigation. Moreover, spillage of irrigation fluid onto the surrounding area, including a health care provider, is prevented thereby helping to reduce biologic contamination.

Some embodiments of the present disclosure therefore relate to a platform for treating a wound, comprising a first side having an island disposed therein and configured to irrigate an extremity, the island surrounded by a fluid collection area; and a second side depending from the first side, the second side having an operating area disposed thereon and a fluid collection channel disposed about the operating area, wherein the first side and the second side are adapted to be reversibly employed, the first side being configured for a first medical procedure and the second side being configured for a second medical procedure. The first side may further include an indentation adapted to receive a distal portion of an extremity. The indentation may be semi-circular in shape and open to the island. The first side may further include multiple walls with the indentation being disposed along one of the walls. A plurality of teeth may extend from at least one of the walls with the teeth being configured to anchor a band.

In this embodiment, the first side may further include a landing area having at least one projection extending in a direction of the island. The projection and the landing area can cooperate with the island to elevate portions of the extremity above the fluid collection channel.

Also in this embodiment, the second side may further include a drainage channel disposed proximate the operating table. The drainage channel may be in communication with the fluid collection channel.

In this embodiment, the first medical procedure may include lavage, disinfection, and the like, and combinations thereof. Likewise, the second medical procedure may include debridement, fracture repair, suturing, and other surgical processes and combinations thereof.

In another embodiment, a medical device for wound treatment may include a first portion having a platform disposed therein and configured to irrigate an appendage wound, the platform disposed proximate a waste collection area, the first portion further including a plurality of walls wherein at least one of the walls has a recess formed therein for receipt of a distal portion of an extremity, at least one of the walls being configured for securing the extremity to the first portion; and a second portion opposite the first portion, the second portion having an operating area formed thereon and a fluid collection channel disposed about the operating area, the second portion being configured for securing the extremity on the operating area, wherein the first and second portions are adapted to be reversibly employed, the first portion being configured for a first procedure and the second portion being configured for a second procedure.

In this embodiment, the first and second portions may be thermoplastic, and at least one of the walls may include a plurality of teeth depending from the wall or walls. The teeth may be configured to receive a band to immobilize the extremity on the platform and/or the operating area.

Also in this embodiment, the system, particularly the first and second portions together, may be from about 9 inches to about 12 inches in length. A width of the first and second portions may be from about 6 inches to about 10 inches. A height of the first and second portions may be from about 2 inches to about 4 inches.

The first procedure in this embodiment may include lavage, disinfection, and other cleansing and surgical preparation operations, and combinations thereof. The second procedure may include debridement, fracture repair, suturing, and other forms of surgery and combinations thereof.

In another aspect of the disclosure, a method for treating a wounded extremity may include providing a platform having an upper portion having a platform disposed therein and configured to irrigate a wound in an extremity, the platform surrounded by a waste collection area, the upper portion including a plurality of walls, at least one of the walls having a recess formed therein for receipt of a portion of the extremity, at least one of the walls including a projection for securing the extremity within the upper portion; and a lower portion having an operating area formed thereon and a fluid collection channel disposed about the operating area, the upper and lower portions being molded together, the projection being further configured for securing the extremity on the operating area of the lower portion, wherein the upper and lower portions are adapted to be reversibly employed, the upper portion being configured for a first procedure and the lower portion being configured for a second procedure. The exemplary may also include performing the first procedure or the second procedure.

The method may also include providing a band or a strap to immobilize the extremity by strapping the band to the projection.

In an exemplary method, the first procedure may include lavage, disinfection, and other pre-surgical preparations and combinations thereof, and the second procedure may include debridement, fracture repair, suturing, and other surgical operations, and combinations thereof. The first procedure may be followed by the second procedure by inverting or reversing the platform.

Additional objects and advantages of the present subject matter are set forth in, or will be apparent to, those of ordinary skill in the art from the description herein. Also, it should be further appreciated that modifications and variations to the specifically illustrated, referenced, and discussed features, processes, and elements hereof may be practiced in various embodiments and uses of the disclosure without departing from the spirit and scope of the subject matter. Variations may include, but are not limited to, substitution of equivalent means, features, or steps for those illustrated, referenced, or discussed, and the functional, operational, or positional reversal of various parts, features, steps, or the like. Those of ordinary skill in the art will better appreciate the features and aspects of the various embodiments, and others, upon review of the remainder of the specification.

### BRIEF DESCRIPTION OF THE DRAWINGS

A full and enabling disclosure of the present subject matter, including the best mode thereof, directed to one of ordinary skill in the art, is set forth in the specification, which makes reference to the appended figures, in which:

FIG. 1 is a perspective view of an irrigation system according to one aspect of the disclosure;

FIG. 2 includes front and rear elevational views of the irrigation system as in FIG. 1;

FIG. 3 includes a plan view and a side elevational view of the system as in FIG. 1;

FIG. 4 is a perspective view of the system as in FIG. 1 in an intended use environment;

FIG. 5 is a perspective view of an irrigation system in an intended use environment according to another aspect of the disclosure; and

FIG. 6 is a side elevational view of the system as in FIG. 5.

### DETAILED DESCRIPTION

Detailed reference will now be made to the drawings in which examples embodying the present subject matter are shown. The detailed description uses numerical and letter designations to refer to features of the drawings.

The drawings and detailed description provide a full and written description of the present subject matter, and of the manner and process of making and using various exemplary embodiments, so as to enable one skilled in the pertinent art to make and use them, as well as the best mode of carrying out the exemplary embodiments. However, the examples set forth in the drawings and detailed descriptions are provided by way of explanation only and are not meant as limitations of the disclosure. The present subject matter thus includes any modifications and variations of the following examples as come within the scope of the appended claims and their equivalents.

The figures are not necessarily to scale, and some features may be exaggerated to show details of particular components. Therefore, specific structural and functional details disclosed herein are not to be interpreted as limiting, but merely as a basis for the claims and as a representative basis for teaching one skilled in the art to variously employ the present invention.

Unless defined otherwise, all technical and scientific terms used herein have the same meaning as is commonly understood by one of ordinary skill in the art to which this disclosure belongs. In the event that there is a plurality of definitions for a term herein, those in this section prevail unless stated otherwise.

Wherever the phrase “for example,” “such as,” “including” and the like are used herein, the phrase “and without limitation” is understood to follow unless explicitly stated otherwise. Similarly “an example,” “exemplary” and the like are understood to be non-limiting.

Any discussion of the prior art throughout the specification should in no way be considered as an admission that such prior art is widely known or forms part of common general knowledge in the field.

It is an object of the present disclosure to overcome or ameliorate at least one of the disadvantages of the prior art, or to provide a useful alternative.

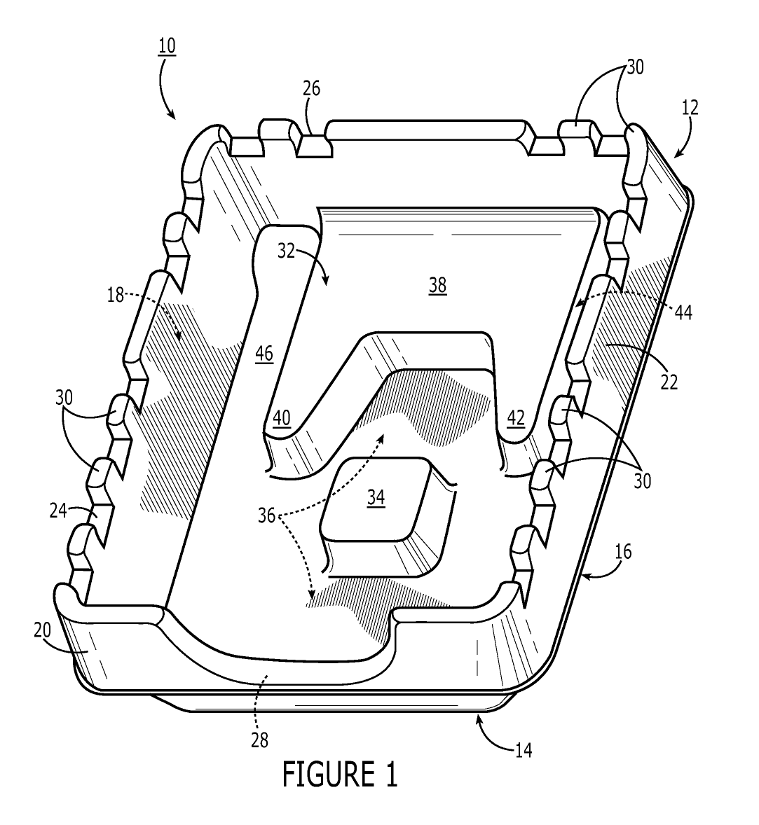

With reference now to the figures, FIG. 1 shows, according to an embodiment of the disclosure, an irrigation, debridement, lavage and/or surgical platform also referred to herein as basin, system, or stand, designated in general by the number 10. The irrigation system 10 may be a Hand Pan® brand platform available from the inventor of the embodiments disclosed herein. Further, the irrigation system 10 may be a moldable thermoplastic, such as an acrylic-polyvinyl chloride composite or high or low density polyethylene, and may broadly include an upper, top or first side or half 12 and a lower, bottom or second side or half 14 forming or having a perimeter, periphery or peripheral wall 16 defining therein a recess, basin, bowl or depression 18. As shown, the peripheral wall 16 may be substantially rectangular in shape having a first side, wall or face 20, a second side, wall or face 22, a third side, wall or face 24, and a fourth side, wall or face 26, although square, oblong, and other shapes may be provided. Here, one or more of the walls 20, 22, 24, 26 may include a cut-out or indentation 28 formed in, on or through the walls 20, 22, 24, 26. In this example, the cut-out 28 is formed in the wall 20 and is semi-circular in shape to receive a human arm or wrist. Also shown, one or more of the walls 20, 22, 24, 26 may include teeth, ridges or projections 30 for receiving straps or bands, as will be explained in detail below.

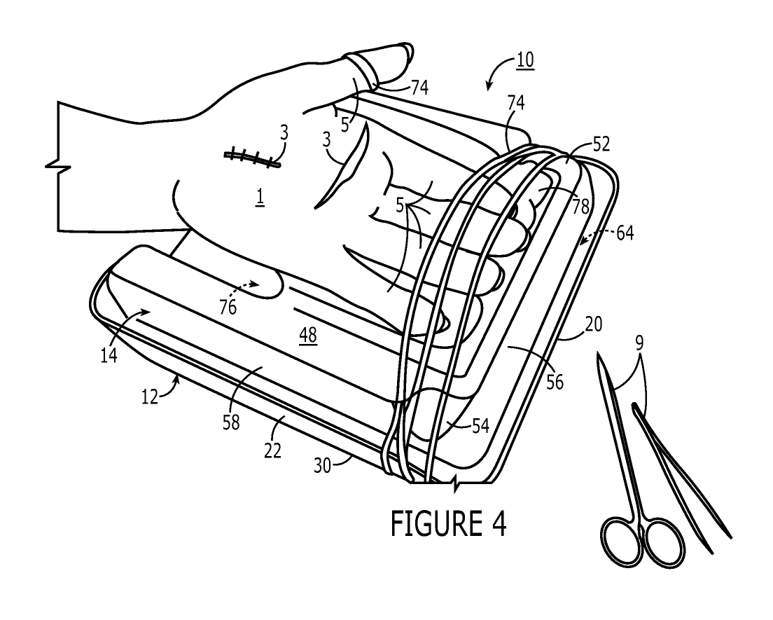

FIG. 1 further shows that within the basin 18, a support member or platform 32 may include a substantially planar area 38 with a first extension or projection 40 and a second extension or projection 42. Here, the projections 40, 42 rise above and extend into a drainage area or fluid/waste collection area 36. Also, a palm or hand platform, island or support stand 34 is set apart from the projections 40, 42, preferably within reach of the projections 40, 42. The stand 34 is substantially surrounded by the waste collection area 36 so that when a palm of a hand, for instance, is placed on the stand 34, fingers of the hand will rest on projections 40, 42 and/or portions of the area 38, as will be described by exemplary operation with respect to FIG. 4 below.

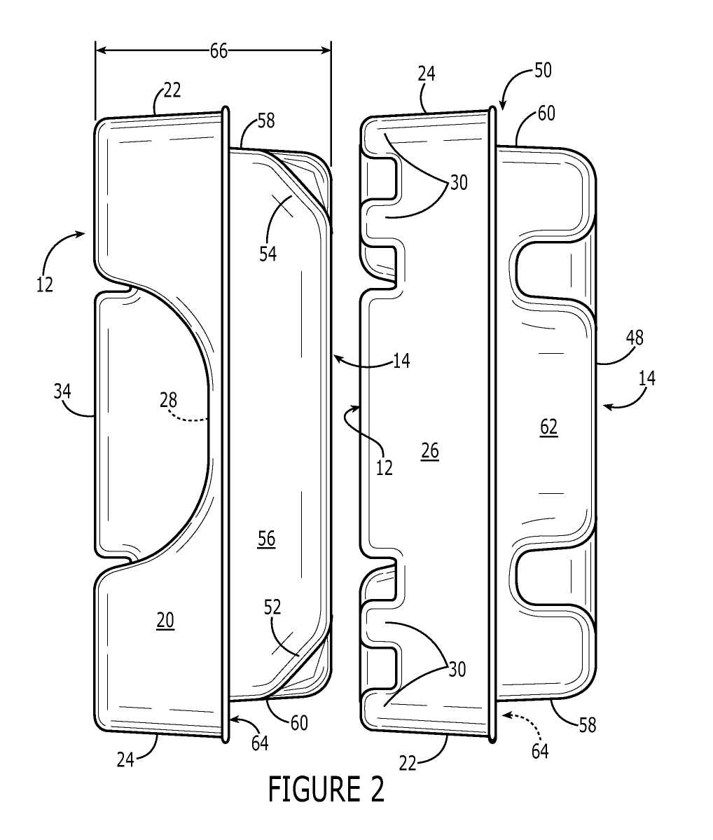

Turning now to FIG. 2, the first end 20 and the opposing end 26 of the irrigation basin 10 are most clearly shown in juxtaposition. Here, a height of the basin 10 is indicated by the numeral 66 and may be from about 2 to about 6 inches, preferably about 3 inches in height. Further, the pocket or wrist holder 28 of the upper half 12, briefly introduced above, is shown formed in the end 20 between walls 22, 24, and a portion of the island 34 may be seen behind the pocket 28. Below the first half 12, the lower half 14 includes a wall 56, which corresponds approximately to the wall 20. Similarly, side walls 58, 60 of lower half 14 approximately correspond to walls 22, 24 of the first half 12. Also shown in FIG. 2 are chamfered or sloped corners or edges 52, 54, which may be used with teeth 30 to help hold a strap in place during operation, as discussed below.

With continued reference to FIG. 2, an irrigation or drainage channel 64 is formed between the walls 20, 22, 24, 26 of the upper half 12 and a periphery 50 formed by the respective walls 56, 58, 60, 62 of the lower half 14. As will be explained below regarding FIG. 4, the channel 64 is used to catch liquids and debris when an operation surface or platform 48 of the lower half 14 is being utilized.

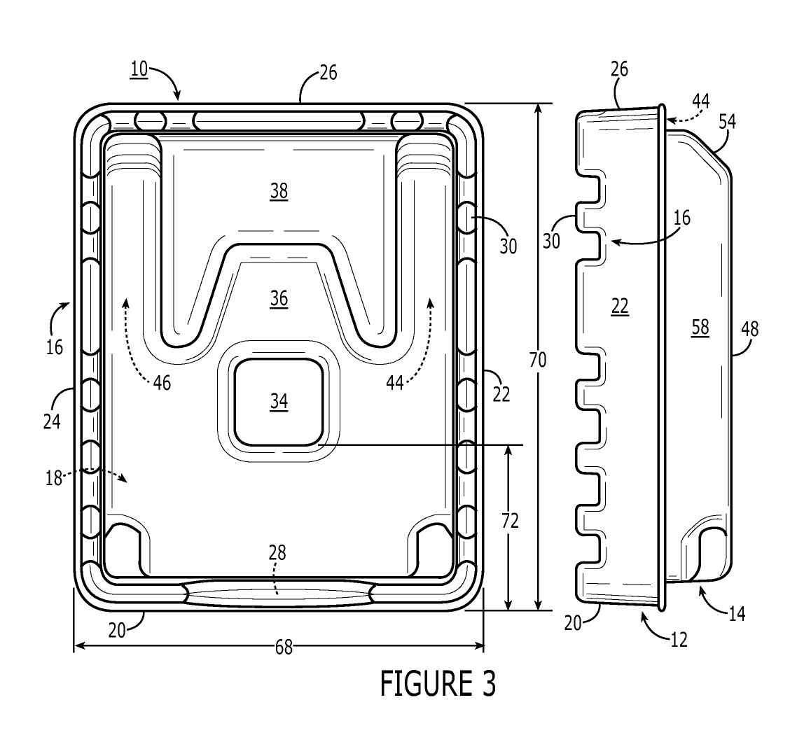

FIG. 3 best shows the periphery 16 of the upper half 12, which includes the walls 20, 22, 24, 26, and the plurality of teeth 30 that will be used to assist in holding a hand stationary. Also as introduced above, shown relative to each other within the basin 18 of the irrigation system 10 are the palm island 34, the fluid/waste collection area 36, the planar area 38, the first extension 40, and the second projection 42. The irrigation channels or gutters 44, 46 are also best shown in FIG. 3.

FIG. 3 shows an overall width of the irrigation system 10 as indicated by the number 68, which may be from about 6 to about 10 inches; preferably about 8 inches in width to best accommodate maneuverability of the system 10 with a hand thereon. Likewise, an overall length of the irrigation system 10 is indicated by the numeral 70 and may be from about 9 to about 12 inches, preferably about 10 inches in length. Also shown, a distance from the wrist opening 28 to a leading edge of the island 34 is indicated by the numeral 72 and may be from about 2 to about 5 inches, preferably about 3.3 inches.

With reference now to FIG. 4, the irrigation system 10, as introduced above, includes the first side 12 and the second side 14. Specifically, the system 10 can be reversibly employed, inverted, or flipped over so that the second side 14 may be used as a work table or surface to facilitate exploration and/or repair of lacerations, wounds, or surgical incisions 3 of a hand 1. The operating face or platform 48 is shown with an optional cushion, pad, absorbent or non-slip surface 78 upon which the hand 1 is positioned. As shown in this exemplary operation, the walls 56, 58 of the second side 14 correspond approximately to respective walls 20, 22 of the first half 12 and form the drainage gutter or moat 64 to catch fluid (see also walls 24, 26, 60, 62 in FIG. 3). Also shown in FIG. 4 are drainage slopes or channels 76 to assist in directing fluid or debris into the channel 64 during an operation or procedure.

FIG. 4 further shows that a thumb and fingers 5 of the hand 1 may be extended and immobilized by straps, rubber bands, strings or the like such as bands 74 connected across, tied to, or anchored by teeth 30 to provide a surgeon 7 with clear access to the wound 3 using, for instance, instruments 9. As shown, the chamfered ends 52, 54 provide notches or angled stops along a portion of the surface 48 to help ensure that the bands 74 remain positioned near the fingertips 5 rather than slipping, in this example, in a direction of the wound 3. This strapping procedure may be particularly necessary if a patient is unconscious or if the hand 1 is numb and needs to be open for a prolonged period, and/or to prevent involuntarily movement and contraction of the hand 1.

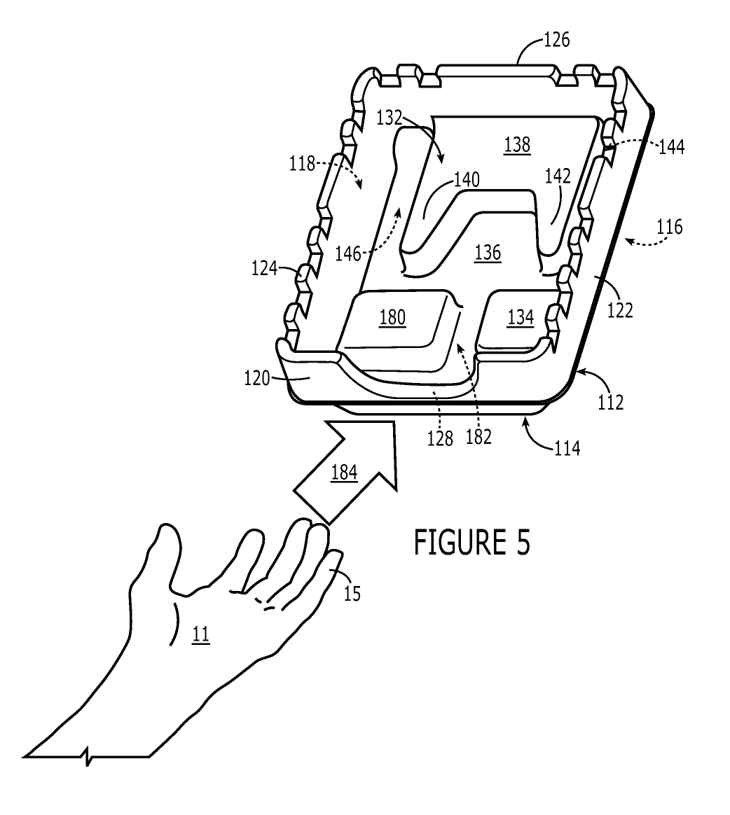

FIG. 5 shows another embodiment of a multipurpose surgical and debridement system, designated in general by element number 110. Here, the system 110 includes a first component or upper operational face 112 and a second component or lower, bottom or second side or operational half 114 for treating an extremity or appendage. The upper face has a perimeter, periphery or peripheral wall 116 defining a recess, basin, bowl or depression 118. As shown, the peripheral wall 116 may be substantially rectangular in shape, although other shapes such as square or oblong may be provided. Here, the periphery 116 may include a first side, wall or face 120, a second side, wall or face 122, a third side, wall or face 124, and a fourth side, wall or face 126. One or more of the walls 120, 122, 124, 126 may include a cut-out, recess, or indentation 128 formed in, on or through the walls 120, 122, 124, 126. In this example, the cut-out 128 is formed in the wall 120 and is semi-circular in shape to receive a human arm or wrist 11 as indicated by an entry arrow 184 in FIG. 5. Also in this example, the perimeter 116, specifically the walls 122, 124, 126, may be substantially smooth or flat as desired for specific operations.

FIG. 5 further shows that within the basin 118, a support member or platform 132 may include a substantially planar area 138 with a first extension or projection 140 and a second extension or projection 142. Here, the projections 140, 142 rise above and extend into a drainage area or fluid/waste collection area 136. Also, a palm or hand platform, island or support stand 134 is set apart from but preferably within reach of the projections 140, 142. Likewise, a second stand 180 is spaced apart from the stand 134 and is substantially surrounded by the waste collection area 136 so that when a palm of the hand 11, for instance, is placed across the stands 134, 180, fingers 15 of the hand 11 will rest on projections 140, 142 and/or upon portions of the area 138 to permit drainage to the area 136 while suitably supporting the hand 11. Likewise, a channel or drainage conduit 182 is formed between stands 134, 180, which in conjunction with channels 144, 146, assists with drainage of fluids or debris into area 136.

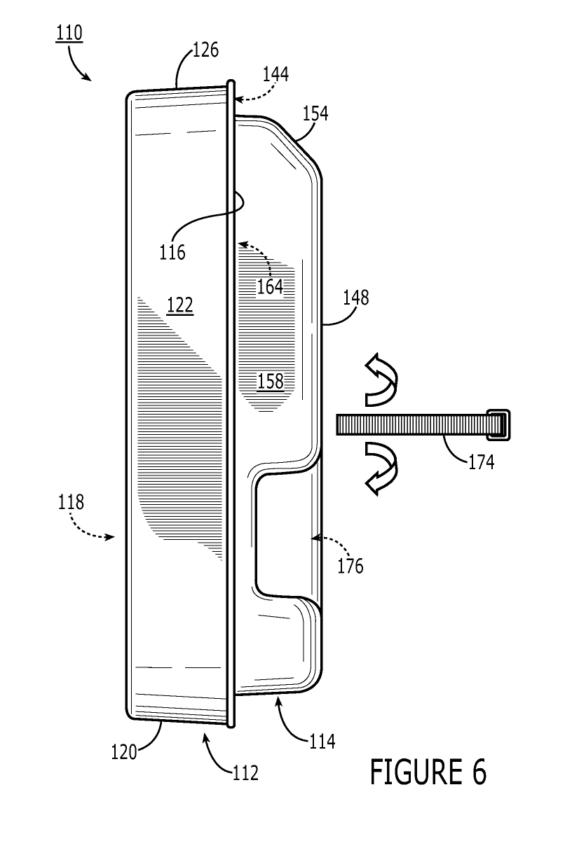

With reference now to both FIGS. 5 and 6, the invertible or multi-use system 110 may include a belt or strap 174 to immobilize the hand 11 either within the basin 118 or upon the platform 148. As shown by the two directional arrows in FIG. 6, the strap 174 may be placed across the hand 11 on the first operational face 112 or on the second operational face 114 depending on which procedure the doctor 7 selects. More specifically, a first procedure might include irrigation, lavage or disinfection, and a second procedure may include debridement, suturing, and/or various types of surgeries, such as to set fractures or to repair tendons or muscles. So, for instance, the doctor 7 might first utilize the operational face 112 for washing the wound 3 in the basin 118 as the first procedure then invert the system 110 to use the operational face 114 to repair a fracture and suture the wound 3 on the platform 148. During the second procedure, as FIG. 6 most clearly shows, the cavity 176 will assist with fluid direction and capture in channel 164.

As previously stated, detailed embodiments of the present invention are disclosed herein; however, it is to be understood that the disclosed embodiments are merely exemplary of the disclosure that may be embodied in various forms. It will be appreciated that many modifications and other variations stand within the intended scope of this disclosure as claimed below. Furthermore, the foregoing description of various embodiments does not necessarily imply exclusion. For example, “some” embodiments may include all or part of “other” and “further” embodiments within the scope of this invention. In addition, “a” does not mean “one and only one;” “a” can mean “one and more than one.”

That which is claimed is:

1. A platform for treating a wound, comprising:
a first side having an island disposed therein and configured to irrigate an extremity, the island surrounded by a fluid collection area; and
a second side depending from the first side, the second side having an operating area disposed thereon and a fluid collection channel disposed about the operating area;
wherein the first side and the second side are adapted to be reversibly employed, the first side being configured for a first medical procedure and the second side being configured for a second medical procedure.

2. The platform of claim 1, wherein the first side further comprises an indentation adapted to receive a distal portion of an extremity, the indentation being semi-circular in shape and opening to the island.

3. The platform of claim 2, wherein the first side further comprises a plurality of walls, the indentation being disposed along one of the walls.

4. The platform of claim 1, wherein the first side further comprises a plurality of walls, a plurality of teeth depending from at least one of the walls and being configured to anchor a band thereto.

5. The platform of claim 1, wherein the first side further comprises a landing area having at least one projection, the projection extending in a direction of the island, the projection and the landing area being configured to operate with the island to elevate portions of the extremity above the fluid collection channel.

6. The platform of claim 1, wherein the second side further comprises a drainage channel disposed proximate the operating table.

7. The platform of claim 6, wherein the drainage channel is in communication with the fluid collection channel.

8. The system of claim 1, wherein the first medical procedure is selected from the group consisting of lavage, disinfection and combinations thereof.

9. The system of claim 1, wherein the second medical procedure is selected from the group consisting of debridement, fracture repair, suturing, and combinations thereof.

10. A medical device for wound treatment, comprising:
a first portion having a platform disposed therein and configured to irrigate an appendage wound, the platform disposed proximate a waste collection area, the first portion further including a plurality of walls wherein at least one of the walls has a recess formed therein for receipt of a distal portion of an extremity, at least one of the walls being configured for securing the extremity to the first portion; and
a second portion opposite the first portion, the second portion having an operating area formed thereon and a fluid collection channel disposed about the operating area, the second portion being configured for securing the extremity on the operating area;
wherein the first and second portions are adapted to be reversibly employed, the first portion being configured for a first procedure and the second portion being configured for a second procedure.

11. The system of claim 10, wherein the first and second portions are thermoplastic.

12. The system of claim 10, wherein at least one of the walls includes a plurality of teeth depending therefrom, the teeth being configured to receive a band to immobilize the extremity on one of the platform and the operating area.

13. The system of claim 10, wherein a length of the first and second portions is from about 9 inches to about 12 inches.

14. The system of claim 10, wherein a width of the first and second portions is from about 6 inches to about 10 inches.

15. The system of claim 10, wherein a height of the first and second portions is from about 2 inches to about 4 inches.

16. The system of claim 10, wherein the first procedure is selected from the group consisting of lavage, disinfection and combinations thereof.

17. The system of claim 10, wherein the second procedure is selected from the group consisting of debridement, fracture repair, suturing, and combinations thereof.

18. A method for treating a wounded extremity, comprising:
providing a platform having:
an upper portion having a platform disposed therein and configured to irrigate a wound in an extremity, the platform surrounded by a waste collection area, the upper portion including a plurality of walls, at least one of the walls having a recess formed therein for receipt of a portion of the extremity, at least one of the walls including a projection for securing the extremity within the upper portion; and
a lower portion having an operating area formed thereon and a fluid collection channel disposed about the operating area, the upper and lower portions being molded together, the projection being further configured for securing the extremity on the operating area of the lower portion;
wherein the upper and lower portions are adapted to be reversibly employed, the upper portion being configured for a first procedure and the lower portion being configured for a second procedure; and
performing one of the first procedure and the second procedure.

19. The method of claim 18, further comprising providing a band and immobilizing the extremity by strapping the band to the projection.

20. The method of claim 18, wherein the first procedure is selected from the group consisting of lavage, disinfection and combinations thereof, and the second procedure is selected from the group consisting of debridement, fracture repair, suturing, and combinations thereof.

21. The method of claim 18, further comprising performing the first procedure followed by the second procedure.
